(19) United States
(12) Plant Patent Application Publication
(10) Pub. No.: US 2004/0068774 P1
(43) Pub. Date: Apr. 8, 2004

(54) "Mann" plum tree

(76) Inventor: Bobby W. Mann, Warrior, AL (US)

Correspondence Address: Wm. Randall May, 1300 Corporate Drive, Birmingham, AL, 35242, US

(21) Appl. No.: 10/263,318

(22) Filed: Oct. 3, 2002

(51) Int. Cl. A01H 5/00

(52) U.S. Cl. PLT/184

(57) ABSTRACT

The present invention relates to a new and distinct variety of Japanese plum tree (Prunus salicina) characterized by its large trunk and limbs, its resistance to disease and by its regular and abundant production of uniformly large and attractive fruit which is mature for harvesting beginning approximately mid-June and which has a distinctive flavor and an excellent storage life.

### BACKGROUND OF THE VARIETY

The present invention relates to a new and distinct variety of plum tree, (Prunus salicina), which will hereinafter be denominated varietally as the “Mann” plum tree, and more particularly to a plum tree which produces, in abundance, a very large, uniform and attractive fruit. The fruit has a distinct flavor and is mature for commercial harvesting approximately mid-June under the ecological conditions prevalent to the north-central portion of the State of Alabama.

### ORIGIN AND ASEXUAL REPRODUCTION OF THE NEW VARIETY

The present variety of the “Mann” plum tree was discovered by the inventor, Bobby W. Mann, in 1988. The new variety was discovered as a chance seedling within the cultivated area of Mr. Mann's orchard in Blount County near Warrior, Ala. The new variety was successfully asexually reproduced by Mr. Mann in 1998 by budding the new variety onto the rootstock of a Halford peach seedling. The resulting asexually reproduced trees have been observed by the inventor since that time and it has been confirmed that the distinctive characteristics of the original parent tree of the new variety are, in all respects, expressed in the asexually reproduced trees.

### SUMMARY OF THE NEW VARIETY

A new and distinct variety of plum tree named the “Mann” plum tree is described. The variety is characterized by its large trunk and limbs, compact growth habit and by the regular production of large, firm, attractive fruit. The fruit produced by this new variety is very large, is fully rounded in shape, has bright yellow-green skin coloration, and possesses an excellent flavor. The large size of the fruit and relative small pit size produces an abundance of edible, good flavored, flesh. It has been determined that the new variety fruit can be harvested beginning approximately mid-June and has an excellent storage life. The asexually reproduced plum tree of the present invention is hearty and will produce a heavy crop of fruit that is easy to harvest. It has also been determined that the blossoms of the variety are frost resistant as compared to other varieties of Japanese plum.

### BRIEF DESCRIPTION OF THE PHOTOGRAPHS

Other distinctive characteristics of the new variety are exemplified in the accompanying color photographs wherein:

FIG. 1 shows an adult parent tree.

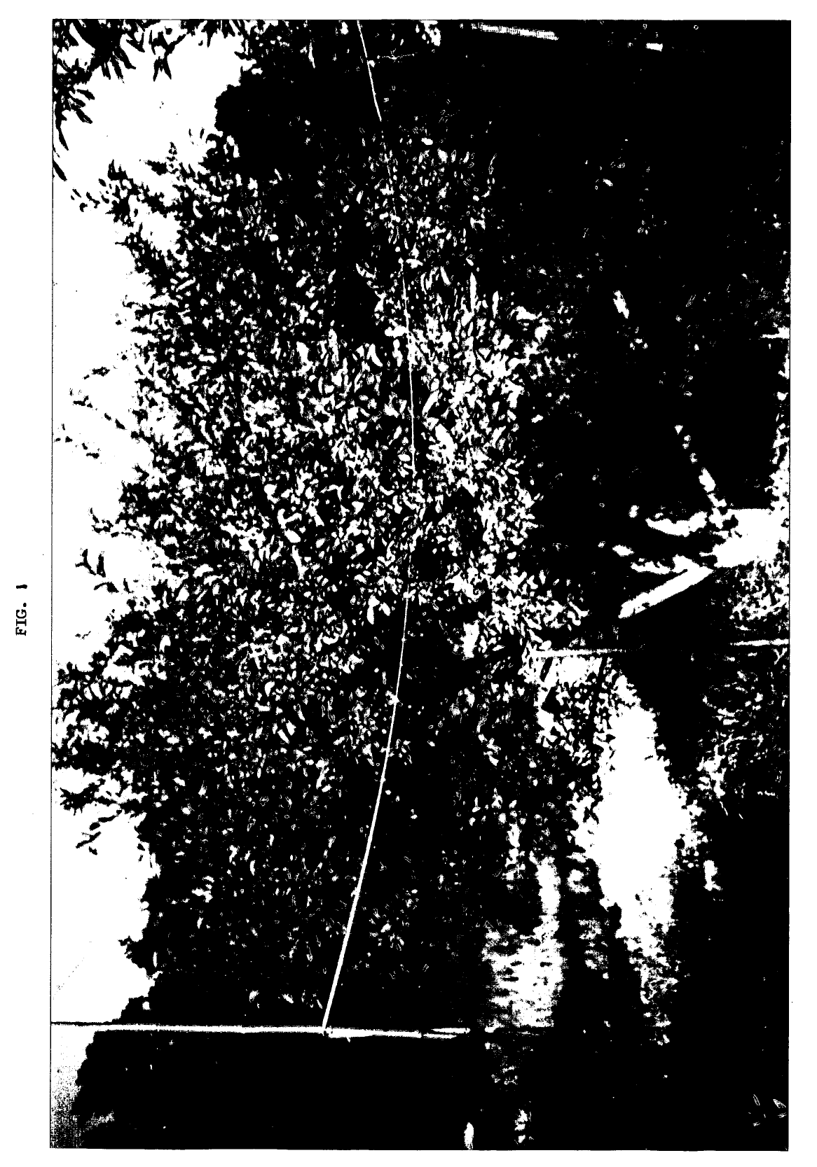

FIG. 2 shows an adult parent tree with adult inflorescence.

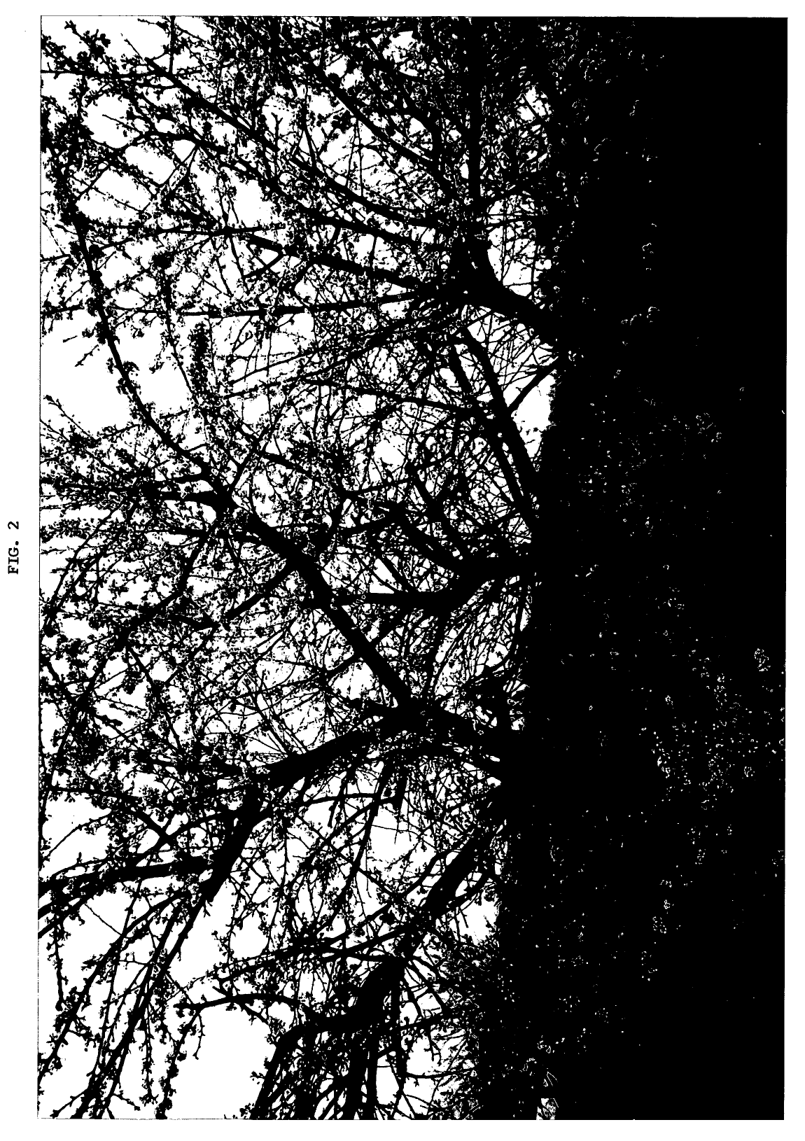

FIG. 3 shows an adult parent tree bearing fruit.

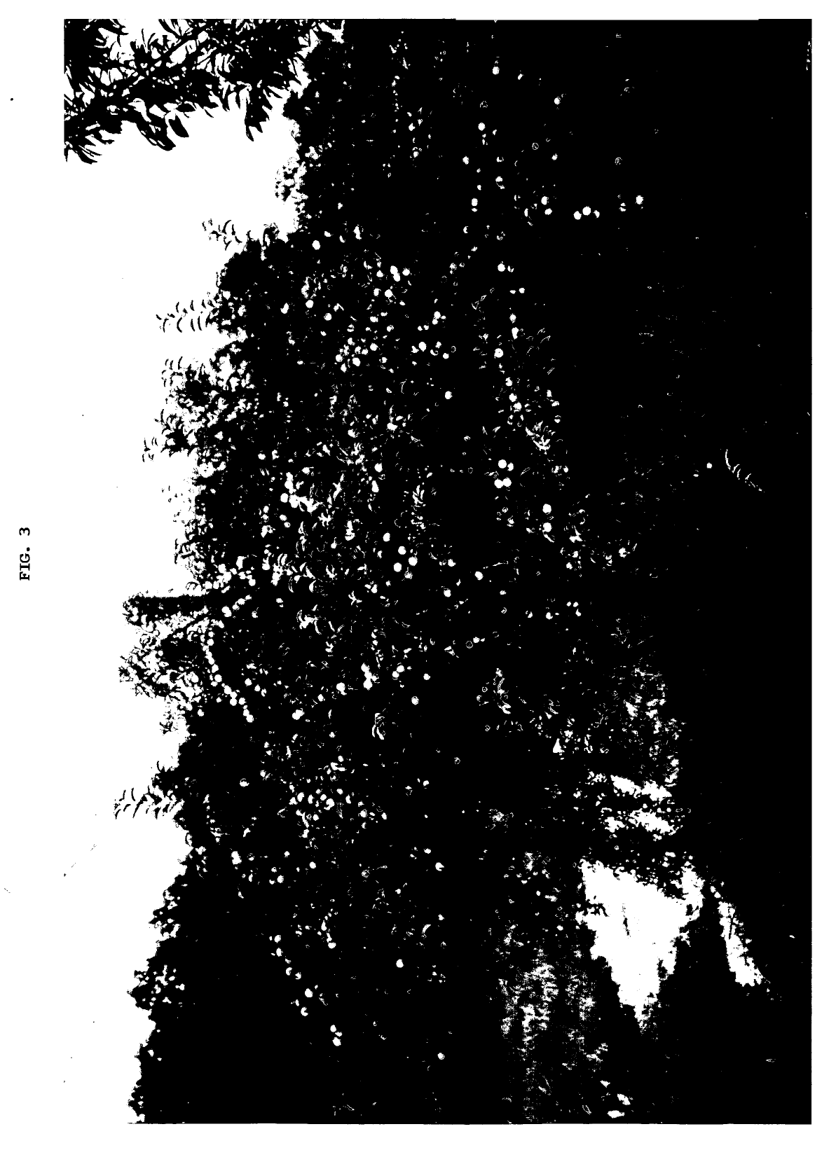

FIG. 4 is a close up of a branch with adult leaves and bearing mature fruit of the new variety.

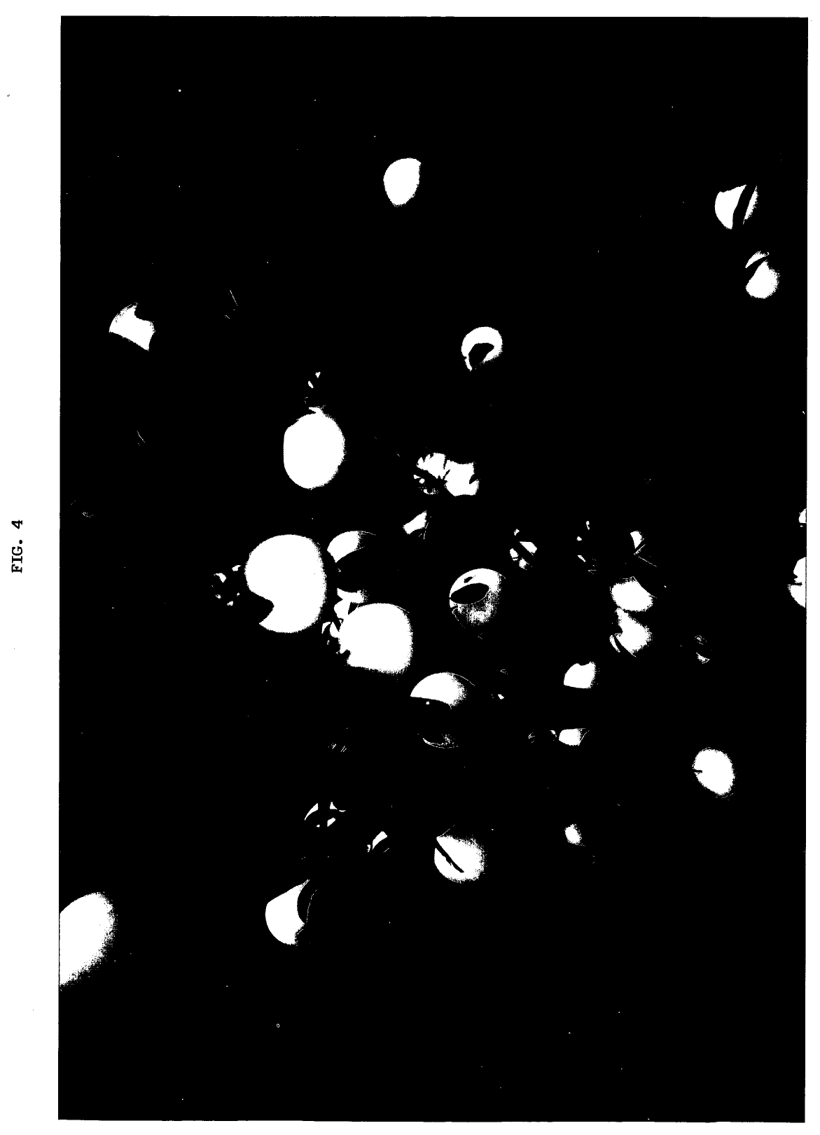

FIG. 5 shows a mature fruit sectioned and laid open with the stone and stem left in place in one section and the stone removed to expose the pit cavity in the other section.

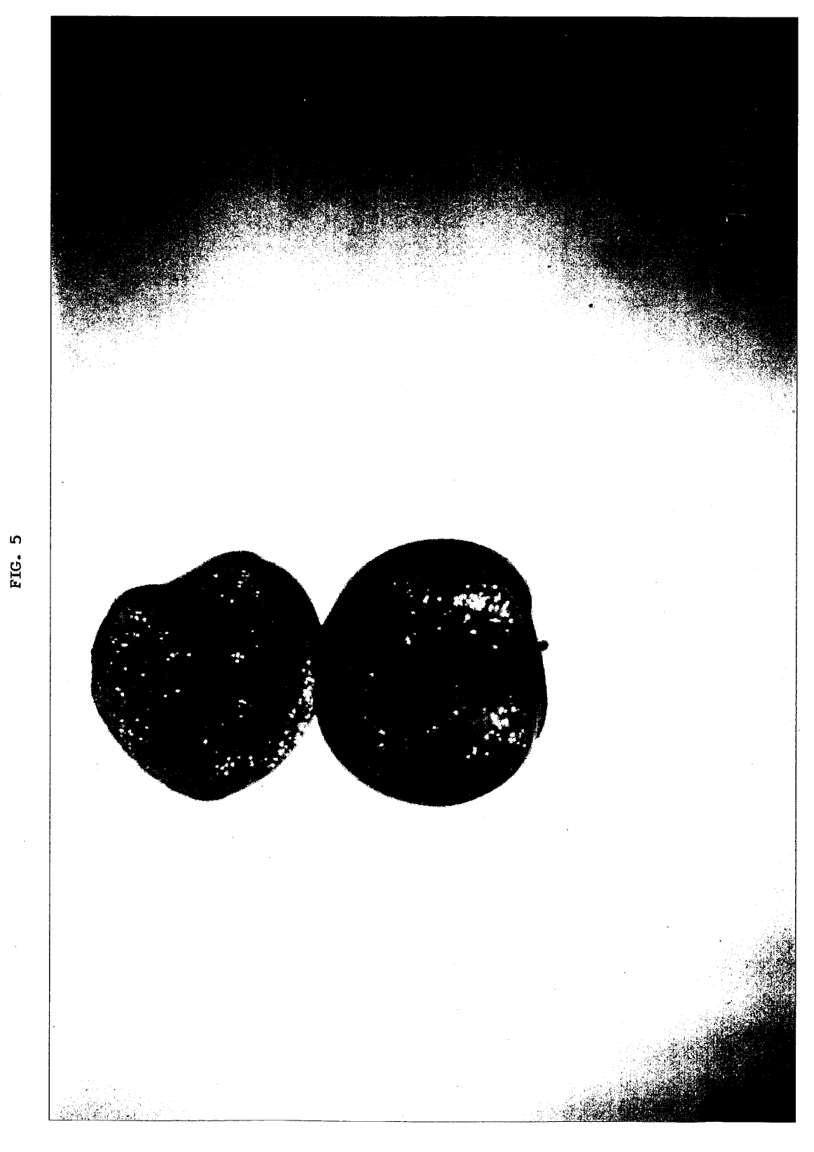

### DETAILED DESCRIPTION OF THE NEW VARIETY

The following is a detailed botanical description of “Mann” plum tree, its flowers, foliage and fruit based on observations of specimens grown in Blount County near Warrior, Ala. at the orchard of origin. All major color-code designations are by reference to the Pantone Book of Color published in 1990 by Harry N. Abrams, Inc., N.Y. Common color names are also occasionally employed.

### TREE

Generally:

Size.—Large and in the upper range for a Japanese plum.

Vigor.—Very vigorous and in the upper range for a Japanese plum.

Chilling requirements.—400 to 750 hours.

Figure.—Bushy tree to height of eleven feet and ten feet wide.

Productivity.—Very productive; large size plum, typically formed in clusters on numerous spurs. Average of 455 pounds per tree in year 2000.

Regularity of bearing.—Regular, good annual crop.

### TRUNK

Size.—Large and in the upper range for a Japanese plum.

Surface.—Smooth to medium rough.

Color.—Sand (Pantone 15-1225).

### BRANCHES

Size.—Medium diameters as scaffold branches are in the medium range for a Japanese plum.

Surface texture.—Mature branches appear slightly rough and immature branches appear smooth.

Lenticels.—Medium number and in the middle range for a Japanese plum.

Color.—Inca Gold (Pantone 17-1048).

### LEAVES

Size.—Large. 107.95 mm length, 44.45 mm wide.

Form/Shape.—Ovate with acuminate tip.

Margin.—Very finely serrated.

Surface.—Upper, reticulate; lower, medium veined.

Thickness.—Medium and in the middle range for a Japanese plum.

Texture.—Glabrous.

Petiole.—Medium size, medium length, medium thickness.

Glands.—No glands found on stem.

Color.—Upper, Cactus (Pantone 18-0130); lower, Jade Green (Pantone 16-0228).

### FLOWER BUDS

Size.—Medium.

Length.—Medium.

Form.—Plump-free.

Surface texture.—Glabrous.

Color.—Lily Green (Pantone 13-0317).

### FLOWERS

Blossom/Bloom period.—About 12 to 14 days. Ranging generally from Mar. 12 to Mar. 26 at Blount County, Alabama.

Size.—Medium.

Pollen.—Semi self-fertile.

Color.—White.

Pistils.—One.

Aroma.—No characteristic fragrance noted.

### FRUIT

Generally:

Maturity.—Fruit matures from the 3^{rd} week of June to approximately July 10 at Blount County, Alabama.

Size.—Medium to large. Average diameter axially 50.5 mm; average diameter transversely in suture plane 49.5 mm.

Weight.—2.5 to 3 ounces each (78 to 93 grams).

Form.—Very good uniformity; shape nearly globose, slightly flattened at stem end.

Suture.—Generally very shallow; inconspicuous line extends from base to the apex and is slightly deeper at cavity.

Base.—Nearly round, slightly flat.

Apex.—Slightly rounded.

Cavity.—Rounded, appx. 5 mm depth.

Stem.—Average length appx. 13 mm.

Ventral surface.—Generally smooth, rounded.

Flesh:

Texture.—Smooth, juicy and crisp at harvest time.

Fibers.—Very small and tender.

Flavor or eating quality.—Flavor — good. Eating quality — good.

Juice.—Slight to moderate.

Aroma.—Slight.

Color.—Cadmium Yellow (Pantone 15-1054).

Ripening.—Even ripening throughout fruit.

Skin:

Thickness.—Medium and in the middle range for a Japanese plum.

Texture.—Medium and in the middle range for a Japanese plum.

Bloom (wax).—Moderate and easily rubbed leaving shiny surface.

Tendency to crack.—None observed.

Taste.—Tart.

Color.—Amber Yellow (Pantone 13-0942).

Stone:

Type.—Cling stone.

Size.— Length appx. 19 mm; thickness appx. 9.5 mm.

Form.—Oval.

Base.—Flat.

Apex.—Very slight point.

Sides.—Generally equal.

Surface.—Relatively smooth and slightly uneven.

Ridges.—None.

Tendency to split.—Not evident.

Color.—Orange Ochre (Pantone 16-1253).

### USE

Fresh fruit market.

### MARKET

Local and long distance.

### KEEPING AND SHIPPING QUALITY

Good to excellent. Fruit remained firm after 14 days out of cool storage.

### RESISTANCE TO DISEASE

Resistance to canker as compared to other varieties of Japanese plum has been noted. No disease observed to date.

Although the new variety of plum tree described herein possesses the described characteristics noted above as a result of the ecological conditions prevailing in Blount County near Warrior, Ala., it is to be understood that these characteristics may vary slightly due to variations in climate, soil conditions, irrigation, fertilization, pruning, pest control, and the like.

What is claimed is:

1. A new and distinct variety of plum tree substantially as illustrated and described, characterized by its large trunk and limbs, compact growth habit and being a regular and productive bearer of uniformly large, firm, attractive clingstone fruit with good flavor and an abundance of edible flesh; the fruit is further characterized by its resistance to disease and by being mature for commercial harvesting and shipment approximately the third week of June under the ecological conditions prevalent to the north-central portion of the State of Alabama.
